(19) United States
(12) Patent Application Publication
(10) Pub. No.: US 2022/0304590 A1
(43) Pub. Date: Sep. 29, 2022

(54) PULSE OXIMETRY DEVICE AND METHOD

(71) Applicant: Analog Devices, Inc., Wilmington, MA (US)

(72) Inventors: Charles MATHY, Arlington, MA (US); Sunrita PODDAR, Jamaica Plain, MA (US); Di XUE, Woburn, MA (US); Miles BENNETT, Stanford, CA (US); Foroohar FOROOZAN, Burlington, MA (US); Edward Joseph DANYLIW, San Francisco, CA (US); Neal Tait KURFISS, Somerville, MA (US)

(73) Assignee: Analog Devices, Inc., Wilmington, MA (US)

(21) Appl. No.: 17/706,196

(22) Filed: Mar. 28, 2022

Related U.S. Application Data
(60) Provisional application No. 63/166,470, filed on Mar. 26, 2021.

(51) Int. Cl. A61B 5/08; A61B 5/1455; A61B 5/00

(52) U.S. Cl. CPC A61B 5/0803; A61B 5/14551; A61B 5/7246; A61B 5/725; A61B 5/6824; A61B 5/6823

(57) ABSTRACT

System and apparatus for pulse oximetry. Systems, methods, and devices are presented for placement of the hardware on a body part where detracting signals are strong. In one example, detracting signals include signals from respiration. In various examples, the body part the oximeter is placed on is a wrist, chest, or arm. A computationally efficient pipeline is presented that allows for reliable and robust SpO2 estimation.

### CROSS REFERENCE TO RELATED APPLICATIONS

This application is related to and claims the benefit of priority under 35 U.S.C. § 119(e) to U.S. Provisional Patent Application No. 63/166,470 entitled “Pulse Oximetry Device and Method” filed on Mar. 26, 2021, which is hereby incorporated oy reference in its entirety.

### FIELD OF THE DISCLOSURE

The present disclosure relates to oximetry. More specifically, this disclosure describes apparatuses and systems for pulse oximeters.

### SUMMARY OF THE DISCLOSURE

Systems, methods, and apparatuses are presented herein for accurate pulse oximetry. In particular, systems and methods are presented for placement of the hardware on a body part where detracting signals are strong. In one example, detracting signals include signals from respiration. In various examples, the body part the oximeter is placed on is a wrist, chest, or arm. A computationally efficient pipeline is presented that allows for reliable and robust SpO2 estimation.

According to one aspect, a method for multichannel pulse oximetry is provided, including collecting data from a plurality of LED-PD channels, assigning a signal quality to each of the plurality of channels, identifying a subset of the plurality of channels with good signal quality, and, for each channel in the subset, estimating heartbeat, extracting a pulsatile component, and estimating SpO2. The method further comprises combining SpO2 estimates from each channel in the subset to generate a final SpO2 estimate.

In some implementations, the method includes determining a final SpO2 estimate accuracy. In some implementations, the method includes determining whether the accuracy exceeds a selected threshold, and, when the accuracy exceeds the selected threshold, reporting the final SpO2 estimate. In some implementations, collecting data includes collecting data periodically. In some implementations, collecting data includes collecting data continuously.

In some implementations, estimating heartbeat includes adaptively estimating heart rate from the collected data. In some implementations, the plurality of LED-PD channels includes a first channel, the first channel includes a green light LED, and wherein estimating heartbeat includes estimating heartbeat using the green light LED of the first channel. In some implementations, estimating SpO2 includes determining SpO2 in the spectral domain. In some implementations, estimating SpO2 includes determining SpO2 in the time domain. In some implementations, the method includes, for each channel in the subset, identifying respiration noise and removing respiration noise.

According to another aspect, a system for pulse oximetry, comprises: a plurality of light emitting diode-photodiode (LED-PD) channels to collect data; a processor for photoplethysmography to: assign a signal quality to each of the plurality of LED-PD channels; identifying a subset of the plurality of LED-PD channels with good signal quality, and for each channel in the subset: estimate heartbeat, extract a pulsatile component, estimate SpO2; and combine SpO2 estimates from each channel in the subset to generate a final SpO2 estimate.

In some implementations, the system includes an adaptive filter to filter out the heartbeat. In some implementations, the adaptive filter is one of a cone filter and a bandpass filter. In some implementations, adaptive filter settings are determined based on the estimated heartbeat. In some implementations, the p orality of LED-PD channels includes a first channel, the first channel includes a green light LED, and wherein the processor is configured to estimate heartbeat using the green light LED of the first channel. In some implementations, the plurality of LED-PD channels includes a first channel, and the first channel includes a green LED, a red LED, and an infrared LED.

According to another aspect, a wearable device for pulse oximetry comprises: a plurality of light emitting diodes (LEDs) to emit light; a plurality of photodiodes to receive reflected LED light, wherein respective LEDs of the plurality of LEDs and respective photodiodes from the plurality of photodiodes generate a plurality of light emitting diode-photodiode (LED-PD) channels to collect data; a plate to hold the LEDs and photodiodes; and a processor coupled to the plate to: assign a signal quality to each of the plurality of LED-PD channels; identifying a subset of the plurality of LED-PD channels with good signal quality, and for each channel in the subset: estimate heartbeat, extract a pulsatile component, estimate SpO2; and combine SpO2 estimates from each channel in the subset to generate a final SpO2 estimate.

In some implementations, the device includes a baffle coupled to the plate, wherein the baffle includes a raised structure between at least two of the plurality of LEDs. In some implementations, the device includes an adaptive filter to filter out the heartbeat, wherein adaptive filter settings are determined based on the estimated heartbeat. In some implementations, the plurality of LED-PD channels includes a first charnel, the first channel includes a green light LED, and wherein the processor is configured to estimate heartbeat using the green light LED of the first channel.

According to another aspect, the present disclosure is an apparatus for multichannel pulse oximetry including a set of green LEDs, as set of red LEDs, a set infrared LEDs, and a set of photodiodes, wherein the photodiodes are positioned around the LEDs.

The drawings show exemplary [Title] circuits and configurations. Variations of these circuits, for example, changing the positions of, adding, or removing certain elements from the circuits are not beyond the scope of the present invention. The illustrated pace detectors, configurations, and complementary devices are intended to be complementary to the support found in the detailed description.

### BRIEF DESCRIPTION OF THE DRAWINGS

The present disclosure is best understood from the following detailed description when read with the accompanying figures. It is emphasized that, in accordance with the standard practice in the industry, various features are not necessarily drawn to scale, and are used for illustration purposes only. Where a scale is shown, explicitly or implicitly, it provides only one illustrative example. In other embodiments, the dimensions of the various features may be arbitrarily increased or reduced for clarity of discussion.

For a fuller understanding of the nature and advantages of the present invention, reference is made to the following detailed description of preferred embodiments and in connection with the accompanying drawings, in which:

FIGS. 1A-1C depicts representations of the components of a pulse oximetry device, according to various embodiments of the disclosure;

FIG. 1D depicts the photons from an LED entering the body and being collected at a PD, according to various embodiments of the disclosure;

FIG. 2 shows example rata that could be collected by pulse oximetry hardware with 2 channels, and 3 wavelengths, according to various embodiments of the disclosure;

FIG. 3 is a graph showing molar extinction coefficients, according to various embodiments of the disclosure;

FIG. 4 depicts an approach of using the ratio of light collection measurements obtained at two different time points, according to various embodiments of the disclosure;

FIG. 5 is a depiction of different contributions to a PPG signal over some time interval of data, according to various embodiments of the disclosure; and

FIG. 6 is a clock diagram showing a processing method, according to various embodiments of the disclosure.

### DETAILED DESCRIPTION

The problem of oximetry is to estimate the fraction of hemoglobin in the bloodstream that is carrying oxygen, also known as blood saturation or SpO2 (Saturation of Peripheral oxygen (O₂)). SpO2 monitoring has many clinical applications and is used routine y as a health indicator. Pulse oximeters typically estimate SpO2 by having light of different wavelengths travel inside a subject and collecting the light afterwards at a photodiode (PD). Obtaining a signal by sending light through biological tissue is called photoplethysmography (PPG). The most common light sources for PPG are light-emitting diodes (LEDs). Some of the wave lengths may be chosen such that they interact differently with oxygenated and deoxygenated hemoglobin. The return signals may then be processed to obtain an estimate of SpO2.

Systems, methods, and apparatuses are presented herein for accurate pulse oximetry. In particular, systems and methods are presented for pulse oximetry hardware that can be placed a body part where detracting signals are strong. In one example, detracting signals include signals from respiration. In various examples, the body part the oximeter is placed on is a wrist, chest, or arm. A computationally efficient pipeline is presented that allows for reliable and robust SpO2 estimation.

The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Other objects, advantages and novel features of the disclosure are set forth in the proceeding in view of the drawings where applicable.

Typical clinical grade pulse oximeters are placed around a fingertip. The light is emitted on one side of the finger and collected on the other, which is an example of what is called transmissive mode pulse oximetry as the light passes through the finger. With the advent of wrist-worn health monitoring devices, the prospect of continuously monitoring SpO2 raises interesting medical possibilities, for example the early detection of health problems before they become severe. Another location for continuous monitoring could be at the chest, a popular placement for health monitoring during exercise. In both cases the light cannot be sent through the body as it would be fully absorbed. Therefore, a reflective mode is the preferred mode of operation for such placements, where the emitters and receivers of light are located dose to each other, and the receivers collect light after it enters the body and may undergo scattering before it returns to a collection point.

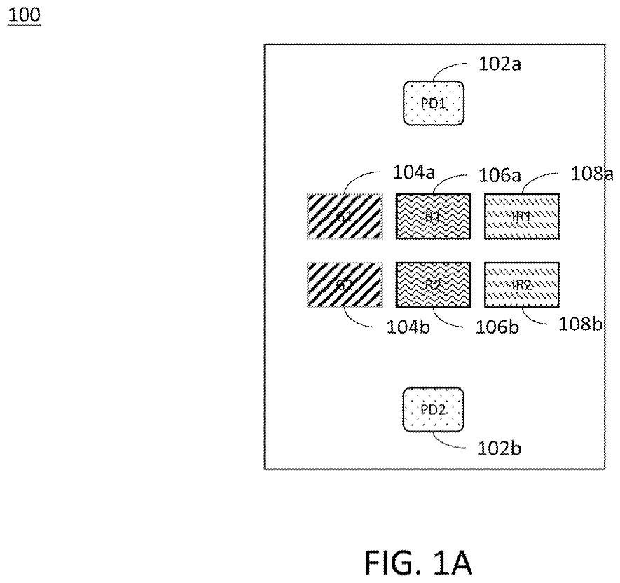

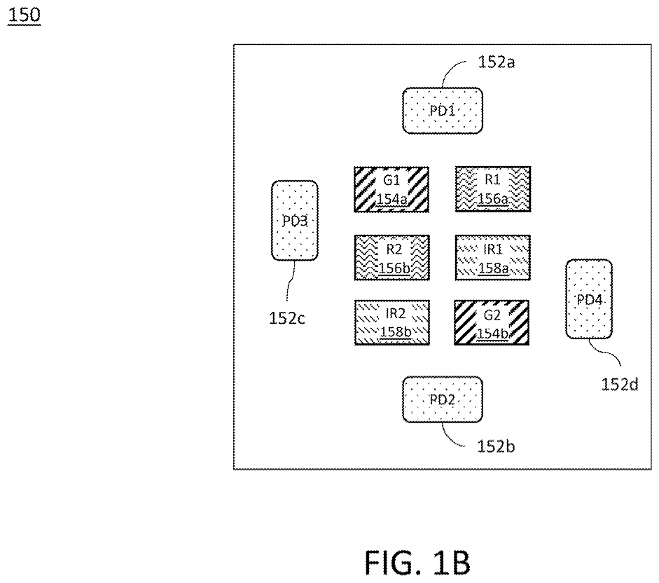

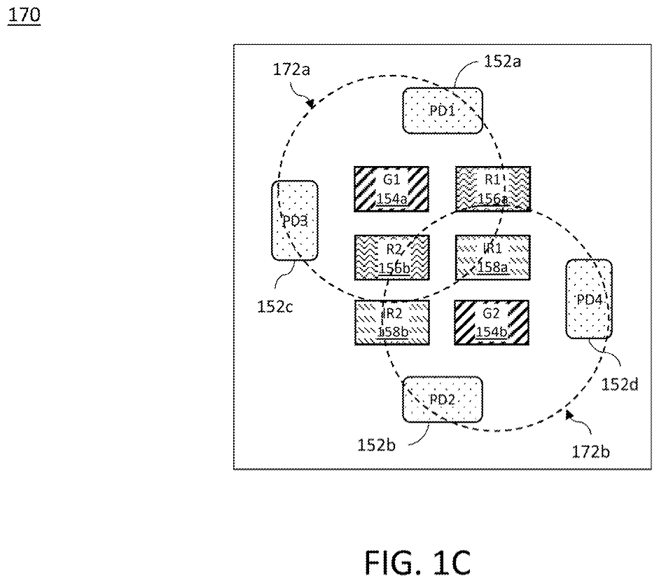

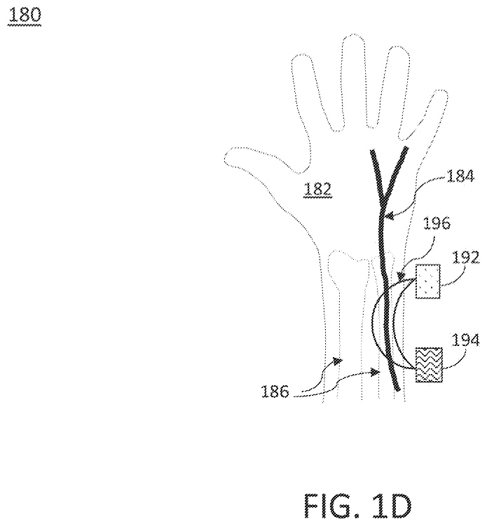

FIG. 1A depicts a diagram 100 of the components of a pulse oximetry device, according to various embodiments of the disclosure. The pulse oximetry device shown in the diagram 100 includes two photodiodes 102a, 102b at opposite sides of the diagram, with light emitting diodes positioned between the photodiodes 102a, 102b. Light-emitting components are referred to herein as light emitting diodes (LEDs), and light-receiving components as photodiodes (PDs), but the LEDs and PDs can be replaced by other components that perform the same basic function (e.g. lasers instead of LEDs). The pulse oximetry device shown in the diagram 100 includes first 104a and second 104b green LEDs, first 106a and second 106b red LEDs, and first 108a and second 108b infrared LEDs, The LEDs 104a, 104b, 106a, 106b, 108a, 108b are positioned between the photodiodes 102a, 102b.

FIG. 1B shows a diagram 150 of a pulse oximetry device, according to various embodiments of the disclosure. The diagram 150 shows four photodiodes 152a, 152b, 152c, 152d surrounding six LEDs. In particular, the pulse oximetry device shown in the diagram 150 includes first 154a and second 154b green LEDs, first 156a and second 156b red LEDs, and first 158a and second 158b infrared LEDs. In some examples, the green LEDs 1541, 154b have a wavelength of about 528 nm. In some examples, the red LEDs 156a, 156b have a wavelength of about 660 nm. In some examples, the infrared LEDs 158a, 158b have a wavelength of about 850 nm. The LEDs 154a, 154b, 156a, 156b, 158a, 158b are positioner between the top 152a and bottom 152b photodiodes and between the left 152c and right 152d photodiodes. When a photodiode is positioned at some distance from an LED, the amount of light collected at the photodiode from the LED decays quickly with distance.

FIG. 1C shows a diagram 170 of the pulse oximetry device shown in FIG. 1B with “splash zones” overlayed on the diagram 170, according to various embodiments of the disclosure. The top left green LED 154a in FIG. 1C is positioned such that the top 152a and left 152c photodiodes collect a lot of light from the top left green LED 154a. In particular, as indicated by the dashed line 172a around the green LED 154a, the top 152a and left 152c photodiodes are in the splash zone of the top left green LED 154a. Additionally, the top left green LED 154a is positioned such that the bottom 152b and right 152d photodiodes won't collect much light from the top left LED 154a. Similarly, the bottom left green LED 154b is positioned such that the bottom 152b and right 152d photodiodes collect a lot of light from the bottom right green LED 154b. In particular, as indicated by the dashed line 172b around the green LED 154b, the bottom 152b and right 152d photodiodes are in the splash zone of the bottom right green LED 154b. Additionally, the bottom right green LED 154b is positioned such that the top 152a and left 152c photodiodes won't collect much light from the bottom right LED 154b. The splash zone for each green LED 152a, 152b is indicated by the dashed lines 172a, 172b surrounding each respective green LED 152a, 152b.

Energy efficiency increases with more photodiodes in the splash zone of an LED. In particular, when there are more photodiodes that are in the splash zone of an LED, the LED can be switched on for less time for a given signal level, because several photodiodes are receiving the signal.

According to various implementations, for pulse oximetry, red and infrared LEDs are positioned at a fixes distance from a photodiode. Thus, the light from the two LEDs travels the same distance to the photodiode, which allows for measurement of absorption of one wavelength vs the other wavelength in the blood. If one wavelength travels through more blood than the other wavelength, that interferes with the comparison. Thus, in the design shown in FIG. 1C, the middle left red 156b and bottom left infrared 158b LEDs are about the same distance from the right photodiode 152d. Similarly, the top right red 156a and middle right infrared LEDs are equidistant from left photodiode 152c. Additionally, the middle left red 156b and middle right infrared 158a LEDs are equidistant from the top 152a and bottom 152b photodiodes. This results in four “channels”, where a channel is a pair of LEDs equidistant from a photodiode. Thus, the center red LED 156b is a part of three different channels (with right 152c, top 152a, and bottom 152b photodiodes). Similarly, the center infrared LED 158a is a part of three different channels (with left 152c, top 152a, and bottom 152b photodiodes). In this manner, there are numerous channels for the various LEDs. This allows the device to be compact and energy efficient, while also letting the light travel in four different directions (left right up coven). Allowing the light to travel in four different directions increases the likelihood of collecting good signals for pulse oximetry, because sometimes one channel couples we to blood while another channel does not.

In some implementations, light is collected from all photodiodes for a given LED. In other implementations, light is collected from a subset of the photodiodes for a given LED. In some examples, the signals collected by all of the photodiodes can be summed. In some examples, the signals are summed directly in hardware, which saves power and memory, since each data stream is not processed. In some implementations, a subset of the signals are summed. In some implementations, each signal is collected independently. If a channel is one LED and one photodiode, then with six LEDs and four photodiodes, there are up to twenty-four channels.

According to various implementations, the wavelengths of the LEDs can be changed. In one example, the top left LED is infrared and the bottom right LED is red. That is, the green LEDs are replaced, resulting in more red-infrared channels. If a top left green LED is replaced with an infrared LED, for example, this results in another red-infrared pair the same distance from the top and bottom photodiodes. Additionally, the top left infrared and left red are then same distance from left photodiode. Thus, replacing just the top left green LED with an infrared LED results in three more channels. Similarly, if the bottom right green LED is replaced with a red LED, for example, this results in another red-infrared pair the same distance from the top and bottom photodiodes. Additionally, the bottom right red and left infrared are then same distance from left photodiode. Thus, replacing just the bottom right green LED with a red LED results in three more channels. In other implementations, the wavelengths of any of the LEDs can be changed.

In some examples, a cone filter is used to filter out the heartbeat. In some examples, a bandpass filter is used to filter out the heartbeat.

In some examples, the green LEDs are uses for heartbeat identification, while the red and infrared LEDs are used for oxygen saturation measurements. In some examples, information received from the green LED signals is used to remove interference from oxygen saturation measurements. For example, heartbeat and respiration noise can interfere with oxygen saturation measurements.

Thus, systems and methods are provided for a pulse oximetry compact design. The design includes multiple photodiodes to increase overall signal collection from the LEDs. For some wavelengths, for examples, wavelengths used to estimate heart rate (e.g. green), having the photodiodes at a distance that collects a lot of light is advantageous for energy and signal level considerations. For some wavelengths, for example wavelengths for which the traveling distance in the body is relevant (e.g. red and infrared to estimate SpO2), having a photodiode at the same distance from the LEDs makes the travel distance in the body more similar. Having multiple photodiodes with the same distance from red and infrared in different orientations can help with determining more accurate SpO2 measurements.

The design is compact, energy efficient, and offers a wide variety of channels from which to estimate blood oxygenation (and possibly other things like heart rate, heart rate variability, blood pressure, etc).

In some implementations, a baffle is used in the pulse oximetry device design. The baffle can be a plate or other structure, and the LEDs and photodiodes can be attached to the baffle. In some examples, the LEDs and photodiodes are attached to a board or plate and the baffle is added to the board. The baffle can be coated with a matte Black paint such that it is opaque and non-reflective. In some examples, glass windows are used for the LEDs and/or photodiodes, and the baffle can minimize reflections and/or other non-signal carrying noise. In some examples, the baffle prevents light from an LED from reflecting to a photodiode without first entering tissue. That is, the baffle helps ensure that light received at a photodiode has first entered the users tissue, such as the users wrist, chest, or other body area.

In various examples, the baffle includes holes and/or windows for each of the LEDs as well as holes and/or windows for each of the photodiodes. In some examples, the baffle includes internal walls to prevent light leaks through adjacent windows. Thus, green light from a first green LED only emits through the first green LEDs respective window. Similarly, red light from a first red LED only emits through the first red LEDs respective window and infrared light from a first infrared LED only emits through the first infrared LEDs respective window. In some examples, each row of LEDs is fully separate, while the columns of LEDs share a window. In some examples, the LEDs in a column of LEDs are partially blocked from each other. In some examples, each column of LEDs is fully separate, while the rows of LEDs share a window. In some examples, the LEDs in a row of LEDs are partially blocked from each other. In some examples columns and/or rows of LEDs are separated by a section of baffle that is about 0.25 mm wide. The baffle can include an overhang.

FIG. 1D is a diagram 180 showing a hand and wrist 182 and depicts the photons from an LED 194 entering the body and being collected at a photodiode 192, according to various embodiments of the disclosure. In particular, as shown in the diagram 180, the hand and wrist 182 include an artery 184 and bones 186. Light from the LED 194 enters the wrist, where it reaches the artery 184 and the bone 186, and is reflected 196 back to the photodiode 192.

As described above, a set of LEDs combined with a photodiode is called a channel. One can potentially collect data for each channel, by turning on respective LEDs, and collecting at the corresponding photodiode. The LEDs need not be turned on simultaneously, if one is looking to collect for each LED independently. If there are N sets of LEDs with the wave lengths that are being used, and M PDs, then there are N×M channels from which one could potentially collect PPG signals.

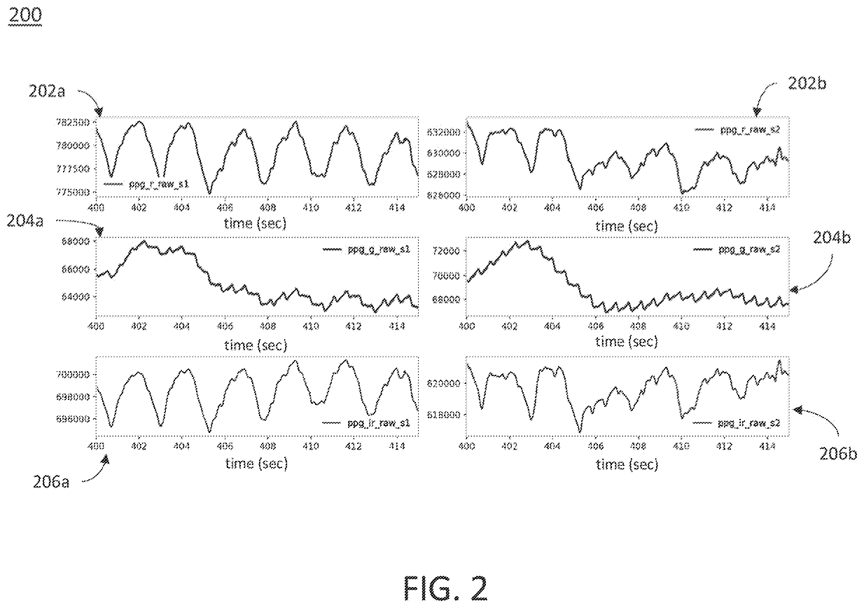

FIG. 2 shows example data 200 that could be collected by pulse oximetry hardware with 2 channels, and 3 wavelengths: green (g), red (r), and infrared (ir), according to various embodiments of the disclosure. In particular, FIG. 2 shows raw PPG rata for hardware with two channels, first channel s1 202a, 204a, 206a, and second channel s2 202b, 204b, 206b, and LEDs of three wavelengths: red (r) 202a, 202b, green (g) 204a, 204b, and infrared (ir) 206a, 206b. For example, the first graph 202a refers to data from the red channel collected at channel 1, the second graph 202b refers to data from the red channel at collected at channel 2, the third graph 204a refers to data from the green channel at collected at channel 1, the fourth graph 204b refers to data from the green channel at collected at channel 2, the fifth graph 206a refers to data from the infrared channel at collected at channel 1, and the sixth graph 206b refers to data from the infrared channel at collected at channel 2.

The extinction coefficient of blood for a given wavelength, which is a measure of how many photons with that wavelength are absorbed by blood, can affect the shape of the PPG signals, if the light travels through Blood in the body. If the wavelength travels through arteries, then as the heart pumps Mood through the arteries, a pulsatile component of the PPG signal may be obtainer, which manifests as either peaks or troughs at the heart rate. How pronounced the pulsatile component is can depend on the wavelength. If a wavelength has a relatively high extinction coefficient for example, then the pulsatile component may be more pronounced. The pulsatile component may be most pronounced at the green wavelength, as the extinction coefficient in blood is larger for green than for red and infrared. For red and infrared PPG data the pulsatile components may be overshadowed by non-pulsatile contributions, for example from respiration.

FIG. 2 depicts the extinction coefficient for oxygenated hemoglobin Hb and deoxygenated hemoglobin HbO2. For green wavelengths (graphs 204a, 204b), which can be around 550 nm, the extinction coefficients are relatively larger than for red (around 650 nm) and infrared (above 700 nm). Thus, the pulsative peaks and troughs may be more pronounced for green PPG signals. In some cases the pulsatile peaks and troughs may be obscured in the red and infrared channels. The reasons for this may be that there are contributions from respiration that can be stronger than the pulsatile component. Respiration may draw some blood in and out the vascular system, and possibly lead to small motion of the muscles and joints in the body, which may appear in the PPG signals.

To develop robust pulse oximetry hardware, one challenge has been to design a system that can reliably extract the pulsatile component from the different channels. Namely, if the pulsatile components of the PPG signals can be extracted, then the theory of pulse oximetry applies, which derives equations for estimating SpO2 from the pulsatile components at wavelengths that are absorbed differently by Hb and HbO2, for example red and infrared wavelengths. For placements such as on the wrist or the chest, contribution from sources such as respiration may be much stronger than at the fingertip, for example, and can lead to large errors in SpO2 estimation. Systems and method are provided herein for a pulse oximetry device that extract the pulsatile components from the different channels.

At the chest, the respiratory contribution can be strongly amplified due to placement on or near the chest cavity. At the wrist, the relatively large number of veins can lead to large respiratory contributions to the PPG signal. In particular, veins can be strongly impacted by respiration due to being a low pressure system that responds to the pressure changes produced at the lungs when breathing. Otherwise, non-pulsatile contributions to the PPG signal can occur due to photons traveling through bone, muscle, and fat. However, pulse oximetry theory removes any contributions to SpO2 that are constant. If, on the other hand the contributions to SpO2 change over time, such as may be the case with contributions due to respiration, the contributions can negatively impact the estimation of the pulsatile component.

In some implementations, an energy efficient system continuously monitors SpO2 while running on a battery. This feature may affect the amount of processing that can occur on an embedded processor.

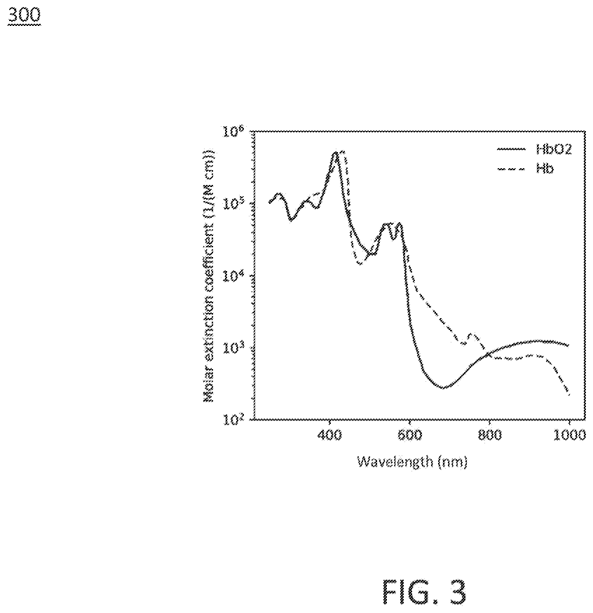

FIG. 3 is a graph 300 showing molar extinction coefficients for deoxygenated hemoglobin Hb (cashed line) and oxygenated hemoglobin HbO2 (solid line) over various wavelengths, according to some embodiments of the disclosure. Wavelengths for which the extinction coefficients are high are strongly absorbed by blood, and therefore a more pronounced pulsatile component in the PPG signal is expected. As shown in the graph 300, shorter wavelengths have higher molar extinction coefficients.

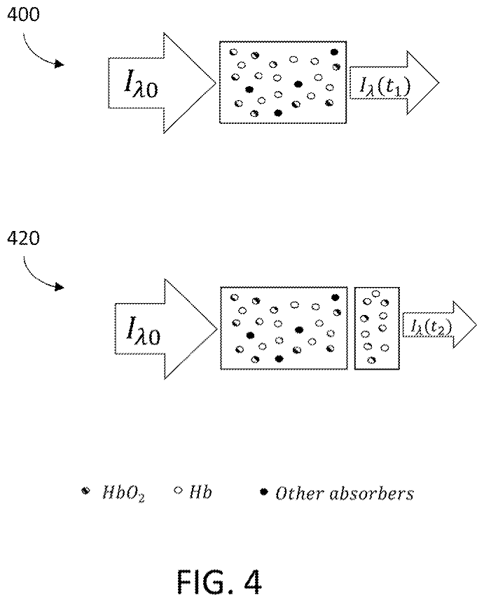

FIG. 4 depicts an approach to determining blood oxygenation by using the ratio of light collection measurements obtained at two different time points 400 and 420, according to various embodiments of the disclosure. As shown in the diagram 400, a PPG signal is acquired at time t₁ by emitting an amount of light I_{λ0} of wavelength λ through a part of the body, and collecting the return light at a light collection point, I₈₀ (t₁) at time t₁. Similarly, as shown in the diagram 420, a PPG signal is acquired at time t₂ by emitting an amount of light I_{λ0} of wavelength λ through a part of the body, and collecting the return light at a light collection point, I_{λ}(t₂) at time t₂.

In the example of PPG signal acquisition shown in FIG. 4, I_{λ0} is the intensity of light of wavelength λ entering the body. At time t₁, an amount of light I(t₁) is collected at a light collection point. At a different time t₂ where the only change is that some extra blood has appeared in the travel path of the light, for example from the blood pumped into arteries by a heart, an amount of light I(t₂) is collected at the same light collection point. If the difference in collected light at wavelength λ between two times t₁ and t₂ is mostly due to added or removed blood, for example Hood pumped through the arteries oar a heart, then the Beer-Lambert law of absorption states that the ratio I_{λ}(t₁)/I_{A}(t₂) will depend mostly on quantities related to the added or removed blood:

I_{λ}(t₁)/I_{λ}(t₂)=exp(−(ϵ_{HbO}_{₂}_{^{λ}}c_{HbO}_{₂}_{^{+ϵ}}_{Hbλ}c_{Hb})d) (1)

where d is the travel distance of the light in the extra blood, c_{HbO}_{₂} (c_{Hb}) is the concentration of HbO₂ (Hb) in the blood, and ϵ_{HbO}_{₂}(ϵ_{Hb}) is the extinction coefficient of HbO₂ (Hb). In this example, the absorption in the blood is dominated by Hb and HbO₂. If such as measurement is carried out at two wavelengths λ₁ and λ₂, then a quantity called the ratio of ratios R may be computed:

$$R = \frac{ln ( \frac{{I}_{{λ}_{1}} ( {t}_{1} )}{{I}_{{λ}_{1}} ( {t}_{2} )} )}{ln ( \frac{{I}_{{λ}_{2}} ( {t}_{1} )}{{I}_{{λ}_{2}} ( {t}_{2} )} )} \qquad ( 2 )$$

By definition the blood saturation SpO2 depends on the concentrations c_{HbO}_{₂} (c_{Hb}) of HbO₂ (Hb) in the blood via the relation SpO2=c_{HbO2}/(c_{Hb}+c_{HbO2}). From equation (1) derived via the Beer-Lambert relation, an equation relating SpO2 to the measured and computed R can be obtained of the form:

$$SpO 2 = \frac{{a}_{1} + {a}_{2} R}{{a}_{3} + {a}_{3} R} \qquad ( 3 )$$

Where a₁,a₂,a₃,a₄ are constants. Empirically, one may estimate the coefficients a₁,a₂,a₃,a₄ from experiments. A different functional form may be found empirical to give a better fit, for example SpO2=a₁+a₂R+a₃R².

According to various implementations, to increase the signal-to-noise ratio, one may take a set of pairs of time points (t₁,t₂), estimate R for these pairs of time points using equation (2), and average the estimations. One approach, which may be referred to as the derivative method, is, for each of the pairs of time points, to use the original time points at which the PPG signals were collected and the next point, (t₁,t₁+Δt) where Δt is the inverse of the sampling rate. If Δt is small enough, then I_{λ}_{ᵢ} can be approximated:

I_{λ}_{ₜ}(t₁+Δt)≈I_{λ}_{ᵢ}(t₁+Δt)+ΔtI′_{λ}_{ᵢ}(t₁) (4)

where I′_{λ}_{ᵢ}(t₁) denotes the time derivative. Then, the ration of time points can be estimated by: In

$$( \frac{{I}_{{λ}_{i}} ( {t}_{1} )}{{I}_{{λ}_{i}} ( {t}_{2} )} ) ≃ - Δ t \frac{I {′}_{{λ}_{i}} ( {t}_{1} )}{{I}_{{λ}_{i}} ( {t}_{1} )} ,$$

and therefore

$$R ≃ \frac{\frac{{I}_{{λ}_{1}}^{′} ( {t}_{1} )}{{I}_{{λ}_{1}} ( {t}_{1} )}}{\frac{{I}_{{λ}_{2}}^{′} ( {t}_{1} )}{{I}_{{λ}_{2}} ( {t}_{1} )}} \qquad ( {1}^{′} )$$

The estimated R can be averaged over a time window, or equation (1′) can be rewritten as:

$$\frac{{I}_{{λ}_{1}}^{′} ( {t}_{1} )}{{I}_{{λ}_{1}} ( {t}_{1} )} ≃ R \frac{{I}_{{λ}_{2}}^{′} ( {t}_{1} )}{{I}_{{λ}_{2}} ( {t}_{1} )} \qquad ( 5 )$$

and a linear fit between

$$\frac{{I}_{{λ}_{1}}^{′} ( {t}_{1} )}{{I}_{{λ}_{1}} ( {t}_{1} )} and \frac{{I}_{{λ}_{2}}^{′} ( {t}_{1} )}{{I}_{{λ}_{2}} ( {t}_{1} )}$$

over a series of time points can be determined. The slope of the linear fit can then be used as an estimate of R.

One important aspect of obtaining SpO2 from applying a function to a measured and computed R is that R should have minimal contributions from quantities outside of the concentrations of Hb and HbO2 in the arterial blood. For example, the venous blood may have a different SpO2, as it may contain blood that has peen depleted of oxygen after going through oxygen consuming tissue or organs. However, if the contribution of the venous blood to the measurement is approximately the same for two times t₁ and t₂, then, as depicted in FIG. 4, the contributions from quantities outside of the concentrations of Hb and HbO2 in the arteria blood will not affect the quantity

$$\frac{{I}_{{λ}_{1}} ( {t}_{1} )}{{I}_{{λ}_{1}} ( {t}_{2} )} ,$$

as any contribution is cancelled in the ratio.

If the difference in absorbers when going from t₁ to t₂ includes elements that are neither Hb nor HbO₂ and contribute significantly to the difference between I_{λ}(t₁) and I_{λ}(t₂), then I_{λ}(t₂)/I_{λ}(t₁) may depend on quantities that are not directly related to concentrations of Hb and HbO₂. In this case, a processor may be applied on I_{λ}(t₁) and I_{λ}(t₂) such that the difference between the processed I_{λ}(t₁) and I_{λ}(t₂) is dominated by contribution from extra blood.

According to various implementations, however, any contribution to a PPG signal that is varying over time and not coming from blood in the arteries (for example, venous blood or moving tissue), will not necessarily be cancelled out by the ratio and may affect the value of R. Any contribution to a

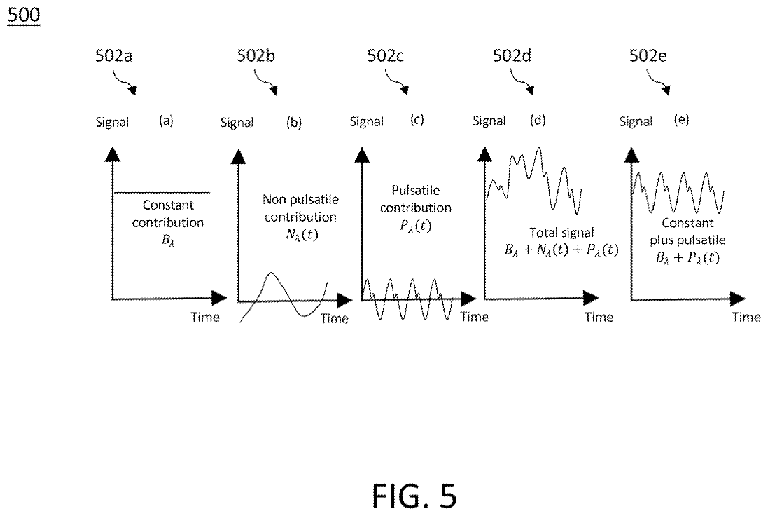

PPG signal that affects the value of R affects the estimated SpO2. Thus, systems and methods are provided for removing time-varying contributions to the PPG signals used to estimate SpO2 that are not due to arterial blood. FIG. 5 illustrates the problem of the contributions to the PPG signals.

FIG. 5 is a diagram 500 showing different non-arterial blood contributions to a PPG signal over a selected time interval of data, according to various embodiments of the disclosure. In various examples, contributions can include a constant contribution as shown in the first graph 502a, a non-pulsatile time-varying contribution as shown in the second graph 502b, and a pulsatile contribution as shown in the third graph 502c. The fourth graph 502d shows a sum of the various contributions shown in the first 502a, second 502b, and third 502c graphs. According to various implementations, the fifth graph 502e shows a reconstructed signal with the non-pulsatile time varying contribution shown in the second graph 502b removed.

### Processing Pipeline

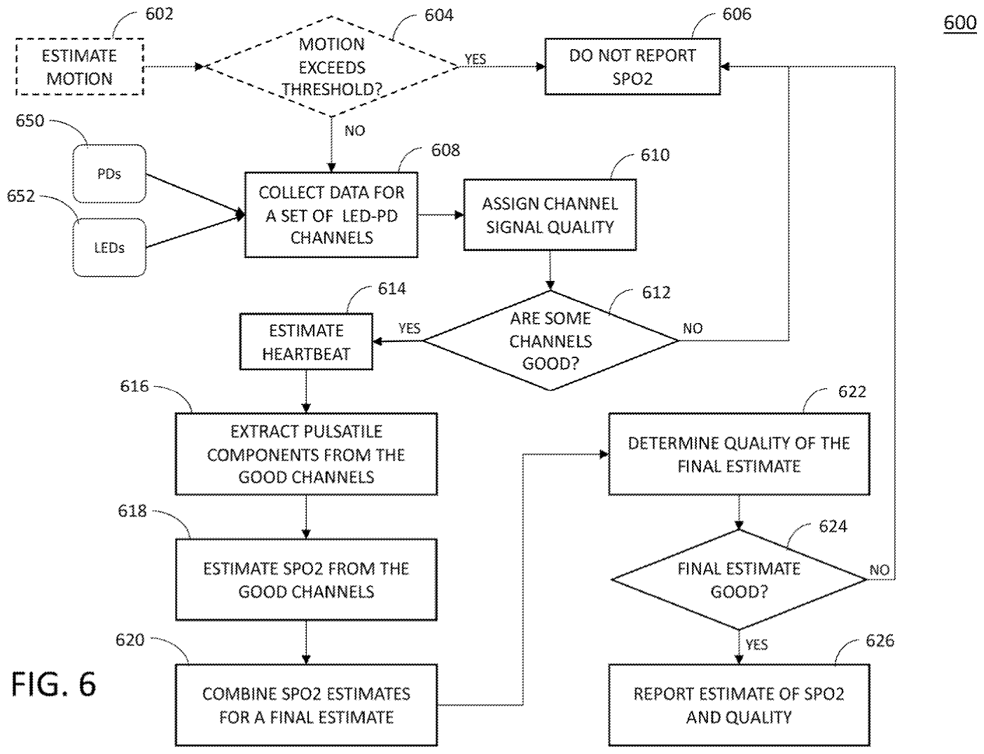

According to various implementations, a processing pipeline uses data collected from one or several channels to estimate SpO2. FIG. 6 is a block diagram showing a processing method 600, according to various embodiments of the disclosure. In some examples, the system is equipped with the ability to estimate motion, and the method 600 begins at step 602. In particular, at step 602, an amount of motion is estimated. For instance, in a system including an accelerometer, motion can be estimated using accelerometer data. In another example, motion is estimated by determining energy in the PPG signals.

At step 604, if the estimated amount of motion from step 602 exceeds a threshold, the method 600 proceeds to step 606, and no SPo2 measurements are reporter. In some examples, at step 606, the system reports that it cannot estimate SPo2 as motion may negatively affect the estimate. At step 604, if the estimated motion does not exceed the threshold, the method proceeds to step 608. At step 608, the system collects data from a set of photodiode 650 and LED 652 channels. For each channel, the system can collect a PPG signal for a set of wavelengths.

According to some examples, to aid in the extraction of the pulsatile components of the PPG signals, the heartbeat is estimated first. For example, PPG data collected during a selected time window can be considered. In some examples, the time window is about 5 seconds, about 8 seconds, about 10 seconds, or longer than about 10 seconds. In some examples, the heartbeat is more pronounced in selected PPG signals, and a PPG signal with a more pronounced heartbeat is identified. In some examples, a PPG signal acquired using a green wavelength near 650 nm is used to estimate the heart rate. In one approach, the fast Fourier transform (FFT) is taken of a PPG signal, and the frequency is obtained that maximizes the amplitude of the FFT. The frequencies of a FFT of a segment of rata of time T are spaced out oy a distance of 1/T, and therefore the frequency resolution of such an approach is 1/T. The heart rate can be adaptively measured, such that it is measured during the selected time window, and is re-measured in a subsequent time window.

In some examples, a finer resolution is desired. However, it may not be possible to simply increase the time T, because increasing the time T can potential y increase the wait time before reporting SpO2. Additionally, as the time T is increased, the heart rate can change, and the method assumes a constant heart rate. However, a FFT can be an efficient way to compute a series of inner products. In particular, the components of a FFT correspond to an inner product of the data with sines and cosines. For example, given a time series of data {x₀,x₁, . . . ,x_{N-1}} where N is the number of data points with corresponding equally spaced time points {t₀,t₁, . . . ,t_{N-1}}, the FFT at frequency f can be represented as a complex number F_{F}=F_{Rf}+iF_{If} where F_{Rf} (the real part) and F_{If} (the imaginary part) are real numbers. F_{Rf} (the real part) and F_{If} (the imaginary part) can be defined by:

$$\begin{matrix}{F}_{Rf} = \frac{1}{\sqrt{N}} {\sum }_{i = 0}^{N - 1} cos ( \frac{2 π f}{T} {t}_{i} ) {x}_{i} & ( 6 ) \\ {F}_{If} = \frac{1}{\sqrt{N}} {\sum }_{i = 0}^{N - 1} sin ( \frac{2 π f}{T} {t}_{i} ) {x}_{i} & ( 7 )\end{matrix}$$

One approach to increasing the resolution of a FFT is to zero-pad the data, i.e. replace {x₀,x₁, . . . ,x_{N-1}} with {x₀,x₁, . . . ,x_{N-1},0, . . . ,0} and perform a FFT on the data with zeros appended to the end. However, in some examples the ensuing FFT is over a larger number of points, which can use more computational power and memory. To increase the resolution of the heart rate, inner products can be used directly as shown in equations (6) and (7), Such inner products do not require much memory, and the computational power may be far less than what is used for a large FFT. A binary search around an initial estimate of the heart rate, as obtained, for example, by finding the maximum amplitude of an initial FFT, can lead to a higher resolution estimation of the heart rate at moderate computational cost. The approach just described can be combined with other techniques, such as downsampling the data to reduce the amount of computation, windowing the cats to reduce spectral leakage, and high-pass filtering to suppress content at lower frequency than the heartbeat (e.g., from respiration).

Using the PPG and SpO2 data, at step 610, a channel signal quality is assigned to each channel. In some examples, the channel signal quality is a numerical value (e.g., on a scale of 1-10, a scale of 1-100, or some other numerical scale). In some examples, the channel signal quality is binary (e.g., good/bad, 1/0). At step 612, it is determined whether any of the channels have a good signal quality. If no channels have a good channel signal quality at step 612, the method proceeds to step 606, and no SpO2 measurement is reported. If one or more channels have a good channel signal quality at step 612, the method proceeds to step 614.

At step 614, heartbeat is estimated. Once the heart rate fₕᵣ is estimated, at step 616, the heart rate can be used to aid an estimation of the pulsatile component of the PPG signals. If the heart rate is constant for a time window, the pulsatile component can be a periodic signal whose frequency content in the Fourier domain is made up of multiples of fₕᵣ, which are the harmonics of a periodic signal. The harmonics can be estimated directly by computing the inner products as per equations (6) and (7) at the multiples of fₕᵣ. For example, the i-th harmonic has frequency i×fₕᵣ, and its components can be computed by replacing f with i×fₕᵣ in equations (6) and (7). Once the harmonics are computed, the pulsatile signal P_{λ}(t) can be reconstructed for any time point t:

$${P}_{λ} ( t ) = \frac{1}{\sqrt{N}} ( {\sum }_{i = 0}^{M - 1} cos ( \frac{2 π f}{T} {t}_{i} ) {F}_{Ri \times {f}_{hr}} - {\sum }_{i = 0}^{M - 1} sin ( \frac{2 π f}{T} {t}_{i} ) {F}_{Ii \times {f}_{hr}} ) \qquad ( 8 )$$

where M is the number of harmonics that have been estimated. Empirically a value for M can be determined that leads to good SpO2 estimation. The time resolution of such a reconstructed pulsatile component is infinite, as it can be evaluated at any time t. The reconstruction of the pulsatile component can be combines with standard techniques such as downsampling and windowing to reduce spectral leakage.

At step 618, SpO2 is estimated from the good channels. Once the pulsatile components have been reconstructed, the ratio of ratios can be estimated. In one example, the derivative method is used. Because the reconstructed signal is a continuous function, a derivative method can be applied at infinite resolution, and an equation can be obtainer for the ratio of ratios that depends only on the quantities F_{Ri×f}_{ₕᵣ} and F_{Ii×f}_{ₕᵣ}. Such an approach offers several advantages. First, it significantly reduces the computational time, because finite differences over a large number of points do are not computed. Second, the sampling rate of the system can be reduced, since the reconstructed signal effectively has infinite time resolution.

There are many potential variations of the approach for reconstruction of the pulsatile component described herein. For example, infinite impulse response filters can be used as computationally efficient alternatives to finite-impulse response filters. In another example, a series of notch filters can be applied at the harmonics of the heartbeat. In a further example, one or more comb filters can be applied at the harmonics of the heartbeat, which let the harmonic stack of the heartbeat through.

At step 620, SpO2 estimates from multiple good channels are combined fora final estimate. In particular, if there are multiple light emission-light collection channels, the measurements from the different channels can be used to obtain a potentially more accurate SpO2 estimate, and combine, for example, with a weighted average:

$$Spo {2}_{final} = {\sum }_{channels} {w}_{i} SpO {2}_{channel i}$$

where wᵢ is the weight assigned to the i-th channel, and Σ_{channels} wᵢ=1. For example, once the pulsatile component has been estimated, the pulsatile component can be removed from the original signal, and subtracting a constant leaves the non-pulsatile time varying component. A channel for which that contribution is relatively large may have a less accurate reconstruction of a pulsatile component. Therefore, a weight that is larger can be used when the estimates non-pulsative time varying component is relatively smaller.

In some examples, a separate estimate of the signal quality can be used for each channel. If the data of given channel is estimated to be low, for example, before the spectral energy at the heartbeat harmonics is relatively small, then it may be assigned a small weight. If it is too small according to some threshold, its weight may be set to zero, which means its estimate of SpO2 is not computed, saving on computation time.

At step 622, the quality of the final estimate is determined. In particular, once the final SpO2 has been estimated, the quality of the estimation can be assessed. At step 624, if the quality is below a selected threshold, the system can decide not to report an SpO2 value and the method 600 proceeds to step 606. At step 624, if the quality is at or above a selected threshold, the method 600 proceeds to step 626 and the estimate of SpO2 is reported along with its quality. The quality assessment can include looking at the level of motion, the signal quality at each channel, and an assessment of the quality of the SpO2 estimation for each channel. For example, if a linear fit is used from the derivative method, the goodness of the fit may be an indicator of the quality of the SpO2 estimate.

### Select Examples

Example 1 provides a method for pulse oximetry, comprising: collecting data from a plurality of light emitting diode-photodiode (LED-PD) channels; assigning a signal quality to each of the plurality of LED-PD channels; identifying a subset of the plurality of LED-PD channels with good signal quality, and for each channel in the subset: estimating heartbeat, extracting a pulsatile component, estimating SpO2; and combining SpO2 estimates from each channel in the subset to generate a final SpO2 estimate.

Example 2 provides a method, system, and/or device according to any of the previous and/or following examples, further comprising determining a fins SpO2 estimate accuracy.

Example 3 provides a method, system, and/or device according to any of the previous and/or following examples, further comprising when the accuracy exceeds a selected threshold, reporting the final SpO2 estimate.

Example 4 provides a method, system, and/or device according to any of the previous and/or following examples, wherein collecting data includes collecting data periodically.

Example 5 provides a method, system, and/or device according to any of the previous and/or following examples, wherein collecting data includes collecting data continuously.

Example 6 provides a method, system, and/or device according to any of the previous and/or following examples, wherein estimating heartbeat includes adaptively estimating heart rate from the collected data.

Example 7 provides a method, system, and/or device according to any of the previous and/or following examples, wherein the plurality of LED-PD channels includes a first channel, the first channel includes a green light LED, and wherein estimating heartbeat includes estimating heartbeat using the green light LED of the first channel.

Example 8 provides a method, system, and/or device according to any of the previous and/or following examples, wherein estimating SpO2 includes determining SpO2 in the spectra domain.

Example 9 provides a method, system, and/or device according to any of the previous and/or following examples, wherein estimating SpO2 includes determining SpO2 in the time domain.

Example 10 provides a method, system, and/or device according to any of the previous and/or following examples, further comprising, for each channel in the subset, identifying respiration noise and removing respiration noise.

Example 11 provides a system for pulse oximetry, comprising: a plurality of light emitting diode-photodiode (LED-PD) channels to collect data; a processor for photoplethysmography to: assign a signal quality to each of the plurality of LED-PD channels; identifying a subset of the plurality of LED-PD channels with good signal quality, and for each channel in the subset: estimate heartbeat, extract a pulsatile component, estimate SpO2; and combine SpO2 estimates from each channel in the sunset to generate a final SpO2 estimate.

Example 12 provides a method, system, and/or device according to any of the previous and/or following examples, further comprising an adaptive filter to filter out the heartbeat.

Example 13 provides a method, system, and/or device according to any of the previous and/or following examples, wherein the adaptive filter is one of a cone filter and a bandpass filter.

Example 14 provides a method, system, and/or device according to any of the previous and/or following examples, wherein adaptive filter settings are determined based on the estimated heartbeat.

Example 15 provides a method, system, and/or device according to any of the previous and/or following examples, wherein the plurality of LED-PD channels includes a first channel, the first channel induces a green light LED, and wherein the processor is configured to estimate heartbeat using the green light LED of the first channel.

Example 16 provides a method, system, and/or device according to any of the previous and/or following examples, wherein the plurality of LED-PD channels includes a first channel, and the first channel includes a green LED, a red LED, and an infrared LED.

Example 17 provides a wearable device for pulse oximetry, comprising: a plurality of light emitting diodes (LEDs) to emit light; a plurality of photodiodes to receive reflected LED light, wherein respective LEDs of the plurality of LEDs and respective photodiodes from the plurality of photodiodes generate a plurality of light emitting diode-photodiode (LED-PD) channels to collect data; a p ate to hold the LEDs and photodiodes; and a processor coupled to the plate to: assign a signal quality to each of the plurality of LED-PD channels; identifying a subset of the plurality of LED-PD charnels with good signal quality, and for each channel in the subset: estimate heartbeat, extract a pulsatile component, estimate SpO2; and combine SpO2 estimates from each channel in the subset to generate a final SpO2 estimate.

Example 18 provides a method, system, and/or device according to any of the previous and/or following examples, further comprising a baffle coupled to the pate, wherein the baffle induces a raised structure between at east two of the plurality of LEDs.

Example 19 provides a method, system, and/or device according to any of the previous and/or following examples, wherein the baffle prevents light from a first LED from reflecting onto one of the plurality of photodiodes without first entering tissue.

Example 20 provides a method, system, and/or device according to any of the previous and/or following examples, further comprising an adaptive filter to filter out the heartbeat, wherein adaptive filter settings are determined based on the estimated heartbeat.

Example 21 provides a method, system, and/or device according to any of the previous and/or following examples, wherein the plurality of LED-PD channels includes a first channel, the first channel includes a green light LED, and wherein the processor is configured to estimate heartbeat using the green light LED of the first channel.

Example 22 provides a method, system, and/or device according to any of the previous and/or following examples, wherein the photodiodes are positioned around the LEDs.

Example 23 provides a method, system, and/or device according to any of the previous and/or following examples, wherein the LEDs includes two red LEDs, two infrared LEDs, and two green LEDs positioned in a 2-by-3 grid.

### Variations and Implementations

Having thus described several aspects and embodiments of the technology of this application, it is to be appreciated that various alterations, modifications, and improvements will readily occur to those of ordinary skill in the art. Such alterations, modifications, and improvements are intended to be within the spirit and scope of the technology described in the application. For example, those of ordinary skill in the art will readily envision a variety of other means and/or structures for performing the function and/or obtaining the results and/or one or more of the advantages described herein, and each of such variations and/or modifications is deemed to be within the scope of the embodiments describe herein.

Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The foregoing outlines features of one or more embodiments of the subject matter disclosed herein. These embodiments are provided to enable a person having ordinary skill in the art (PHOSITA) to better understand various aspects of the present disclosure. Certain well-understood terms, as well as underlying technologies and/or standards may be references without being described in detail. It is anticipated that the PHOSITA will possess or have access to background knowledge or information in those technologies and standards sufficient to practice the teachings of the present disclosure.

The PHOSITA will appreciate that they may readily use the present disclosure as a basis for designing or modifying other processes, structures, or variations for carrying out the same purposes and/or achieving the same advantages of the embodiments introduces herein. The PHOSITA will also recognize that such equivalent constructions do not depart from the spirit and scope of the present disclosure, and that they may make various changes, substitutions, and alterations herein without departing from the spirit and scope of the present disclosure.

The above-described embodiments may be implemented in any of numerous ways. One or more aspects and embodiments of the present application involving the performance of processes or methods may utilize program instructions executable by a device (e.g., a computer, a processor, or other device) to perform, or control performance of, the processes or methods.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above.

The computer readable medium or media may be transportable, such that the program or programs stored thereon may be loaded onto one or more different computers or other processors to implement various ones of the aspects described above. In some embodiments, computer readable media may be non-transitory media.

Note that the activities discussed above with reference to the FIGURES which are applicable to any integrated circuit that involves signal processing (for example, gesture signal processing, video signal processing, audio signal processing, analog-to-digital conversion, digital-to-analog conversion), particularly those that can execute specialized software programs or algorithms, some of which may be associated with processing digitized real-time data.

In some cases, the teachings of the present disclosure may be encoded into one or more tangible, non-transitory computer-readable mediums having stored thereon executable instructions that, when executed, instruct a programmable device (such as a processor or DSP) to perform the methods or functions disclosed herein. In cases where the teachings herein are embodied at least party in a hardware device (such as an ASIC, IP block, or SoC), a non-transitory medium could include a hardware device hardware-programmed with logic to perform the methods or functions disclosed herein. The teachings could also be practiced in the form of Register Transfer Level (RTL) or other hardware description language such as VHDL or Verilog, which can be used to program a fabrication process to produce the hardware elements disclosed.

In example implementations, at least some portions of the processing activities outlined herein may also be implemented in software. In some embodiments, one or more of these features may be implemented in hardware provided external to the elements of the disclosed figures, or consolidated in any appropriate manner to achieve the intended functionality. The various components may include software (or reciprocating software) that can coordinate in order to achieve the operations as outlined herein. In still other embodiments, these elements may include any suitable algorithms, hardware, software, components, modules, interlaces, or objects that facilitate the operations thereof.

Any suitably-configured processor component can execute any type of instructions associated with the data to achieve the operations detailed herein. Any processor disclosed herein could transform an element or an article (for example, data) from one state or thing to another state or thing. In another example, some activities outlined herein may be implemented with fixed logic or programmable logic (for example, software and/or computer instructions executed by a processor) and the elements identified herein could be some type of a programmable processor, programmable digital logic (for example, an FPGA, an erasable programmable read only memory (EPROM), an electrically erasable programmable read only memory (EEPROM)), an ASIC that includes digital logic, software, code, electronic instructions, flash memory, optical disks, CD-ROMs, DVD ROMs, magnetic or optical cards, other types of machine-readable mediums suitable for storing electronic instructions, or any suitable combination thereof.

In operation, processors may store information in any suitable type of non-transitory storage medium (for example, random access memory (RAM), read only memory (ROM), FPGA, EPROM, electrically erasable programmable ROM (EEPROM), etc.), software, hardware, or in any other suitable component, device, element, or object where appropriate and based on particular needs.

Further, the information being tracked, sent, received, or stored in a processor could be provided in any database, register, table, cache, queue, control list, or storage structure, based on particular needs and implementations, all of which could be referenced in any suitable timeframe.

Any of the memory items discussed herein should be construed as being encompassed within the broad term ‘memory.’ Similarly, any of the potential processing elements, modules, and machines described herein should be construed as being encompassed within the broad term ‘microprocessor’ or ‘processor.’ Furthermore, in various embodiments, the processors, memories, network cards, buses, storage devices, related peripherals, and other hardware elements described herein may be realized by a processor, memory, and other related devices configured by software or firmware to emulate or virtualize the functions of those hardware elements.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer out with suitable processing capabilities, including a personal digital assistant (FDA), a smart phone, a mobile phone, an iPad, or any other suitable portable or fixed electronic device.

Also, a computer may have one or more input and output devices. These devices can be used, among other things, to present a user interface. Examples of output devices that may be used to provide a user interface include printers or display screens for visual presentation of output and speakers or other sound generating devices for audible presentation of output. Examples of input devices that may be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wine area network, such as an enterprise network, and intelligent network (IN) or the Internet. Such networks may be based on any suitable technology and may operate according to any suitable protocol and may include wireless networks or wired networks.

Computer-executable instructions may be in many forms, such as program modules, executed by one or more computers or other devices. Generally, program modules induce routines, programs, objects, components, data structures, etc. that performs particular tasks or implement particular abstract rata types. Typically, the functionality of the program modules may be combined or distributed as desired in various embodiments.

The terms “program” or “software” are used herein in a generic sense to refer to any type of computer code or set of computer-executable instructions that may be employed to program a computer or other processor to implement various aspects as described above. Additionally, it should be appreciated that according to one aspect, one or more computer programs that when executed perform methods of the present application need not reside on a single computer or processor, but may be distributed in a modular fashion among a number of different computers or processors to implement various aspects of the present application.

Also, data structures may be stores in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code may be executed on any suitable processor or collection of processors, whether provided in a single computer or distributed among multiple computers.

Computer program logic implementing all or part of the functionality described herein is embodied in various forms, including, but in no way limiter to, a source cote form, a computer executable form, a hardware description form, and various intermediate forms (for example, mask works, or forms generated by an assembler, compiler, linker, or locator). In an example, source code includes a series of computer program instructions implemented in various programming languages, such as an object code, an assembly language, or a high-level language such as OpenCL, RTL, Verilog, VHDL, Fortran, C, C++, JAVA, or HTML for use with various operating systems or operating environments. The source code may define and use various data structures and communication messages. The source code may be in a computer executable form (e.g., via an interpreter), or the source code may be converter (e.g., via a translator, assembler, or compiler) into a computer executable form.

In some embodiments, any number of electrical circuits of the FIGURES may be implemented on a board of an associated electronic device. The board can be a general circuit board that can hold various components of the internal electronic system of the electronic device and, further, provide connectors for other peripherals. More specifically, the board can provide the electrical connections by which the other components of the system can communicate electrically. Any suitable processors (inclusive of digital signal processors, microprocessors, supporting chipsets, etc.), memory elements, etc. can be suitably coupled to the board based on particular configuration needs, processing demands, computer designs, etc.

Other components such as external storage, additional sensors, controllers for audio/video display, and peripheral devices may be attached to the board as plug-in cards, via cables, or integrated into the board itself. In another example embodiment, the electrical circuits of the FIGURES may he implemented as standalone modules (e.g., a device with associated components and circuitry configured to perform a specific application or function) or implemented as plug in modules into application-specific hardware of electronic devices.

Note that with the numerous examples provided herein, interaction may be described in terms of two, three, four, or more electrical components. However, this has been done for purposes of clarity and example only. It should be appreciated that the system can be consolidated in any suitable manner, Along similar design alternatives, any of the illustrated components, modules, and elements of the FIGURES may be combined in various possible configurations, all of which are clearly within the broad scope of this disclosure.

In certain cases, it may be easier to described one or more of the functionalities of a given set of flows by only referencing a limited number of electrical elements. It should be appreciated that the electrical circuits of the FIGURES and its teachings are readily scalable and can accommodate a large number of components, as well as more complicate/sophisticated arrangements and configurations. Accordingly, the examples provided should not limit the scope or inhibit the broad teachings of the electrical circuits as potentially applied to a myriad of other architectures.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

### Interpretation of Terms

All definitions, as defined and used herein, should be understood to control over dictionary definitions, definitions in documents incorporated by reference, and/or ordinary meanings of the defined terms. Unless the context clearly requires otherwise, throughout the description and the claims:

“comprise,” “comprising,”and the like are to be construed in an inclusive sense, as opposes to an exclusive or exhaustive sense; that is to say, in the sense of “including, but not limited to”.

“connected,” “coupled,” or any variant thereof, means any connection or coupling, either direct or indirect, between two or more elements; the coupling or connection between the elements can be physical, logical, or a combination thereof.

“herein,” “above,” “below,” and words of similar import, when used to describe this specification shall refer to this specification as a whole and not to any particular portions of this specification.

“or,” in reference to a list of two or more items, covers all of the following interpretations of the word: any of the items in the list, all of the items in the list, and any combination of the items in the list.

the singular forms “a”, “an” and “the” also include the meaning of any appropriate plural forms.

Words that indicate directions such as “vertical”, “transverse”, “horizontal”, “upward”, “downward”, “forward”, “backward”, “inward”, “outward”, “vertical”, “transverse”, “left”, “right”, “front”, “back”, “top”, “bottom” “below”, “above”, “under”, and the like, used in this description and any accompanying claims (where present) depend on the specific orientation of the apparatus described and illustrated. The subject matter described herein may assume various alternative orientations. Accordingly, these directional terms are not strict defined and should not be interpreted narrowly.

The indefinite articles “a” and “an,” as used herein in the specification and in the claims, unless clearly indicates to the contrary, should be understood to mean “at least one.”

The phrase “and/or,” as used herein in the specification and in the aims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined.

Elements other than those specifically identified by the “and/or” clause may optionally be present, whether related or unrelated to those elements specifically identified. Thus, as a non-limiting example, a reference to “A and/or B”, when uses in conjunction with open-ended language such as “comprising” may refer, in one embodiment, to A only (optionally including elements other than B); in another embodiment, to B only (optionally inducing elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optional y be present other than the elements specifically identifies within the list of elements to which the phrase “at least one” refers, whether relates or unrelated to those elements specifically identified.

Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at east one, optionally including more than one, A, and at least one, optionally including more than one, B (and optionally including other elements); etc.

As used herein, the term “between” is to be inclusive unless indicated otherwise. For example, “between A and B” includes A and B unless indicated otherwise.

Also, the phraseology and terminology used herein is for the purpose of description and should not be regarded as limiting. The use of “inducing,” “comprising,” or “having,” “ containing, involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be dosed or semi-closed transitional phrases, respectively.

Numerous other changes, substitutions, variations, alterations, and modifications may be ascertained to one skilled in the art and it is intended that the present disclosure encompass all such changes, substitutions, variations, alterations, and modifications as falling within the scope of the appended claims.

In order to assist the United States Patent and Trademark Office (USPTO) and, additionally, any readers of any patent issued on this application in interpreting the claims appended hereto, Applicant wishes to note that the Applicant: (a) does not intend any of the appended claims to invoke 35 U.S.C. § 112(f) as it exists on the date of the filing hereof unless the words “means for” or “steps for” are specifically used in the particular claims; and (b) does not intend, by any statement in the disclosure, to limit this disclosure in any way that is not otherwise reflected in the appended claims.

The present invention should therefore not be considered limited to the particular embodiments described above. Various modifications, equivalent processes, as well as numerous structures to which the present invention may be applicable, will be readily apparent to those skilled in the art to which the present invention is directed upon review of the present disclosure.

What is claimed is:

1. A method for pulse oximetry, comprising:
collecting data from a plurality of light emitting diode-photodiode (LED-PD) channels;
assigning a signal quality to each of the plurality of LED-PD channels;
identifying a subset of the plurality of LED-PD channels with good signal quality, and for each channel in the subset:
estimating heartbeat,
extracting a pulsatile component,
estimating SpO2; and
combining SpO2 estimates from each channel in the subset to generate a final SpO2 estimate.

2. The method of claim 1, further comprising determining a final SpO2 estimate accuracy.

3. The method of claim 2, further comprising, when the accuracy exceeds a selected threshold, reporting the final SpO2 estimate.

4. The method of claim 1, wherein collecting data includes collecting data periodically.

5. The method of claim 1, wherein collecting data includes collecting data continuously.

6. The method of claim 1, wherein estimating heartbeat includes adaptive y estimating heart rate from the collected data.

7. The method of claim 6, wherein the plurality of LED-PD channels includes a first channel, the first channel includes a green light LED, and wherein estimating heartbeat includes estimating heartbeat using the green light LED of the first channel.

8. The method of claim 1, wherein estimating SpO2 includes determining SpO2 in the spectral domain.

9. The method of claim 1, wherein estimating SpO2 includes determining SpO2 in the time domain.

10. The method of claim 1, further comprising, for each channel in the subset, identifying respiration noise and removing respiration noise.

11. A system for pulse oximetry, comprising:
a plurality of light emitting diode-photodiode (LED-PD) channels to collect data;
a processor for photoplesmography to:
assign a signal quality to each of the plurality of LED-PD channels;
identifying a subset of the plurality of LED-PD channels with good signal quality, and for each channel in the subset:
estimate heartbeat,
extract a pulsatile component,
estimate SpO2; and
combine SpO2 estimates from each channel in the subset to generate a final SpO2 estimate.

12. The system of claim 11, further comprising an adaptive filter to filter out the heartbeat.

13. The system of claim 12, wherein the adaptive filter is one of a cone filter and a bandpass filter.

14. The system of claim 12, wherein adaptive filter settings are determined based on the estimated heartbeat.

15. The system of claim 11, wherein the plurality of LED-PD channels includes a first channel, the first channel includes a green light LED, and wherein the processor is configured to estimate heartbeat using the green light LED of the first channel.

16. The system of claim 11, wherein the plurality of LED-PD channels includes a first channel, and the first channel includes a green LED, a red LED, and an infrared LED.

17. A wearable device for pulse oximetry, comprising:
a plurality of light emitting diodes (LEDs) to emit light;
a plurality of photodiodes to receive reflected LED light, wherein respective LEDs of the plurality of LEDs and respective photodiodes from the plurality of photodiodes generate a plurality of light emitting diode-photodiode (LED-PD) channels to collect data;
a plate to hold the LEDs and photodiodes; and
a processor coupled to the plate to:
assign a signal quality to each of the plurality of LED-PD channels;
identifying a subset of the plurality of LED-PD channels with good signal quality, and for each channel in the subset:
estimate heartbeat,
extract a pulsatile component,
estimate SpO2; and
combine SpO2 estimates from each channel in the subset to generate a final SpO2 estimate.

18. The device of claim 17, further comprising a baffle coupled to the pate, wherein the baffle includes a raised structure between at least two of the plurality of LEDs.

19. The device of claim 17, further comprising an adaptive filter to filter out the heartbeat, wherein adaptive filter settings are determined cased on the estimated heartbeat.

20. The device of c aim 19, wherein the plurality of LED-PD channels includes a first channel, the first channel includes a green light LED, and wherein the processor is configured to estimate heartbeat using the green light LED of the first channel.
